(19) United States
(12) Patent Application Publication
(10) Pub. No.: US 2021/0228712 A1
(43) Pub. Date: Jul. 29, 2021

(54) HEPATITIS B VACCINE

(71) Applicants: TOKYO METROPOLITAN INSTITUTE OF MEDICAL SCIENCE, Tokyo (JP); NATIONAL UNIVERSITY CORPORATION EHIME UNIVERSITY, Ehime (JP); KAGOSHIMA UNIVERSITY, Kagoshima (JP); BEACLE INC, Kyoto (JP)

(72) Inventors: Michinori Kohara, Tokyo (JP); Takahiro Sanada, Tokyo (JP); Yoichi Hiasa, Ehime (JP); Kyoko Kohara, Kagoshima (JP); Yasumasa Goh, Sakyo-ku (JP); Yasunori Oda, Kyoto (JP)

(21) Appl. No.: 16/630,811

(22) PCT Filed: Jul. 13, 2018
(86) PCT No.: PCT/JP2018/027412

§ 371 Date: Apr. 9, 2021

(30) Foreign Application Priority Data
Jul. 14, 2017 (JP) 2017-138047

(51) Int. Cl. A61K 39/29; A61P 31/20

(52) U.S. Cl. CPC A61K 39/292; A61K 2039/6018; A61P 31/20

(57) ABSTRACT

A hepatitis B vaccine comprising a surface antigen particle that has only hepatitis B virus L protein or a variant thereof assembling on a lipid membrane to form the particle.

### FIELD OF THE INVENTION

The present invention relates to a hepatitis B vaccine using HBs-L antigen.

### BACKGROUND ART

Hepatitis B virus (HBV) has three types of surface antigens, namely, L antigen (consisting of Pre-S1, Pre-S2 and S regions), M antigen (consisting of Pre-S2 and S regions) and S antigen (consisting only of S region) (these antigens are also referred to as HBs-L antigen, HBs-M antigen and HBs-S antigen, respectively). Majority of hepatitis B vaccines use the S antigen and some use the M antigen.

Among the proteins that function as the HBV surface antigens, the Pre-S1 region serves as a sensor for the HBV virus to recognize and bind to human hepatocytes. Therefore, an antibody that can neutralize the function of the Pre-S1 region is not only a promising prophylactic vaccine for hepatitis B but also important as a therapeutic vaccine in that it can inhibit growth of the HBV virus inside the body.

A gene coding for the L protein (also called the L antigen gene) has three translation start sites and a common stop codon. Therefore, when the L antigen gene is expressed in animal cells such as CHO cells, three types of proteins, i.e., L, M and S, are formed. These three types of proteins are displayed on a single lipid particle to form an antigen particle having a mixture of the L, M and S proteins.

Under such circumstances, vaccines that utilize a mixture of the three antigens, i.e., the L, M and S antigens, are also known (for example, Sci-B-Vac™ (VBI Vaccines Inc. Israel), a commercially available prophylactic vaccine). In addition, there is also an idea of utilizing a mixture of the three antigens as a therapeutic vaccine for hepatitis B (“An Hbv vaccine and a process of preparing the same”, Japanese Unexamined Patent Application Publication (translation of PCT) No. 2010-516807).

These antigens, however, are simply a mixture of the L, M and S antigens, and development of a vaccine that only uses the L antigen is not yet known.

Meanwhile, number of people with persistent hepatitis B virus (HBV) infection is estimated to be about 400,000,000 worldwide, where the HBV infection rate in our country reaches 1.5%. In our country, various preventive measures including prevention of mother-to-child transmission of HBV, screening of blood for transfusion, and vaccine administration to high-risk groups (selective vaccination), worked out successfully, and number of people infected with HBV has been decreasing. On the other hand, since a number of people who were left out of these preventive measures lack immunity against HBV and thus are vulnerable to HBV, they could be patients of still existing acute hepatitis B and fulminant hepatitis which are caused by horizontally transmitted primary infection. In order to prevent such horizontal transmission, implementation of universal vaccination against HBV started in our country this year.

As described above, the three types of proteins, namely, HBs-L, HBs-M and HBs-S antigens, exist on the surface of a HBV virus particle (FIG. 1). While two types of prophylactic HBV vaccines are used in our country, both of them uses the HBs-S antigen, where about 10% of people show no HBs antibody production with either vaccines (HB vaccine non-responders) and thus cannot receive the benefit of the HBV vaccination. Therefore, for eradication of horizontal transmission of HBV, a stronger HBV vaccine that has less non-responders is needed. Furthermore, although immunotherapies for hepatitis B have also been made with the use of the I-Ms-S antigen, their therapeutic effects have been insufficient and thus a stronger immunotherapeutic method is required.

### PRIOR ART DOCUMENTS

### Patent Document

Patent document 1: Japanese Unexamined Patent Application Publication (translation of PCT) No. 2010-516807

### Non-Patent Documents

Non-patent document 1: Averhoff F, et al., Am J Prey Med. 1998; 15:1-8.

Non-patent document 2: Horiike N, et al., Hepatol Res. 2002; 23:38-47.

### SUMMARY OF INVENTION

### Problem to be Solved by the Invention

Currently available vaccines use the HBs-S antigen for the sake of manufacturing convenience. However, since the N-terminus of the L protein is responsible for the attachment to the hepatocytes, it is desirable that an antibody or cell-mediated immunity against this region is induced.

Furthermore, if the viral activity is retained after the infection, the chronic hepatitis develops to cirrhosis, hepatocellular carcinoma and to liver failure. Currently, pegylated IFN and nucleotide analog entecavir are used for the treatment of hepatitis B.

Pegylated IFN has immuno stimulating action and antiviral action. While the effect can be maintained highly efficiently in seroconversion cases, it is associated with a significant problem of highly frequent and numerous side effects. Moreover, although entecavir can reduce the amount of HBV DNA by inhibiting the viral replication, its drug efficacy rapidly eliminates by stopping the administration, which can lead to recurrence of hepatitis. Due to such problems, there is a strong need for development of a novel therapy that has a mechanism different from the conventional therapies.

Accordingly, the present invention has an objective of providing a novel therapeutic vaccine for hepatitis B.

The present inventors have gone through intensive investigation to solve the above-described problems and tried to develop a vaccine that shows a stronger effect of preventing onset of infection as compared to the currently available vaccines by immunizing with the HBs-L antigen developed and manufactured by Beacle Inc. (FIG. 2). As a result, the present inventors succeeded in solving the aforementioned problems by use of the HBs-L antigen, thereby accomplishing the present invention.

### Means for Solving Problem

Thus, the present invention is as follows.
(1) A hepatitis B vaccine comprising a surface antigen particle that has only hepatitis B virus L protein or a variant thereof assembling on a lipid membrane to form the particle.
(2) The vaccine according to (1), wherein the L protein or the variant thereof is protein (a) or (b) below:

(a) a protein comprising the amino acid sequence represented by SEQ ID NO:1; and

(b) a protein comprising an amino acid sequence obtained by deleting or substituting a total of 16 or less amino acids of the amino acid sequence represented by SEQ ID NO:1, which are: 6 or less amino acids in the Pre-S1 region represented by the 6th-113rd amino acids; 6 or less amino acids in the Pre-S2 region represented by the 114th-162nd amino acids; and 13 or less amino acids in the S region represented by the 163rd-385th amino acids.
(3) The vaccine according to either one of (1) and (2), wherein the L protein or the variant thereof is expressed in yeast.
(4) The vaccine according to any one of (1)-(3), which produces an antibody against the Pre-S1 and/or PreS2 region of the L protein upon administration to the subject.
(5) The vaccine according to any one of (1)-(4), which induces cell-mediated immunity against the Pre-S1 and/or PreS2 region of the L protein upon administration to the subject.
(6) The vaccine according to any one of (1)-(5), further comprising a core protein of hepatitis B virus.
(7) The vaccine according to (6), which further induces an antibody against the core protein upon administration to the subject.
(8) The vaccine according to either one of (6) and (7), which further induces cell-mediated immunity against the core protein upon administration to the subject.
(9) The vaccine according to any one of (1)-(8), wherein the titer of the neutralizing antibody against hepatitis B virus is at least 2 to 1000.
(10) The vaccine according to any one of (1)-(9), whose effect of inhibiting the binding of hepatitis B virus to human hepatocytes is at least 50-100%.

### EFFECT OF THE INVENTION

The present invention provides a hepatitis B vaccine. For the first time, the present invention succeeded in quantitatively analyzing and showing the titer of a neutralizing antibody against hepatitis B virus to clearly show its anti-hepatitis B virus effect.

### BRIEF DESCRIPTION OF THE DRAWINGS

FIG. 1 A view showing the structure of HBV.

FIG. 2 Views showing the L antigen.

FIG. 3 Views showing silver staining of the L antigen, and western blot using antibodies against S, Pre-S1 and Pre-S2 of the L antigen.

FIG. 4 A diagram showing a method of immunizing tupaia with a HBV antigen.

FIG. 5 A diagram showing a method of immunizing a rabbit with a HBV antigen.

FIG. 6 A diagram showing the results from ELISA for detecting an anti-HBs-L antibody.

FIG. 7 A diagram showing the results from ELISA for detecting an anti-HBs-S antibody.

FIG. 8 A diagram showing the results from ELISA for detecting antibodies in rabbits (immunized with the HBs-S antigen and the HBs-L antigen).

FIG. 9 A diagram showing the titers of the neutralizing antibodies in immunized tupaia sera.

FIG. 10 A diagram showing the activities of the anti-HBs-L antibody and the anti-HBs-S antibody to inhibit hepatocyte binding by HBV.

FIG. 11 A view showing the electrophoresis results of the purified HBcAg.

FIG. 12 Diagrams showing results from ELISA for detecting antibodies in mice.

FIG. 13 A diagram showing the results from testing activation of cell-mediated immunity upon administration of the HBs-L and HBc antigens.

### MODE FOR CARRYING OUT THE INVENTION

Herein, L protein refers to a protein that constitutes the L antigen, M protein refers to a protein that constitutes the M antigen, and S protein refers to a protein that constitutes the S antigen.

The present invention relates to a hepatitis B vaccine using a HBs-L antigen. While a HBs-S antigen is used as a prophylactic vaccine against hepatitis B, about 10% of the people do not show HBs antibody production even upon administration of this vaccine (HB vaccine non-responders), and cannot prevent infection. In addition, although treatments by internal use of a nucleotide analog formulation and interferon therapies have been proposed as antiviral therapies for hepatitis B, the nucleotide analog therapy cannot be stopped and needs to be continued for the rest of the life once the internal use begins while the interferon therapy is associated with numerous side effects. Moreover, it is hard to achieve HBV antigen/antibody seroconversion even when both therapies are employed. There have also been attempts to employ immunotherapies using the HBs-S antigen of hepatitis B in the past, but their therapeutic effects have been insufficient. Therefore, in order to prevent hepatitis B virus infection, the present invention aims at developing a prophylactic vaccine using the HBs-L antigen that differs from and has stronger immunization action than the currently available vaccines and at developing an immunotherapy using this antigen as a therapeutic vaccine.

The present inventors studied the possibility of a hepatitis B vaccine that uses particles displaying only L antigen, and found that an effect superior than conventional vaccines can be expected, thereby succeeded in accomplishing the present invention.

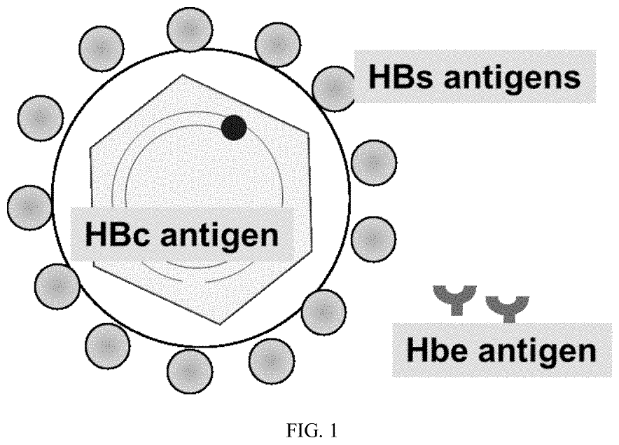

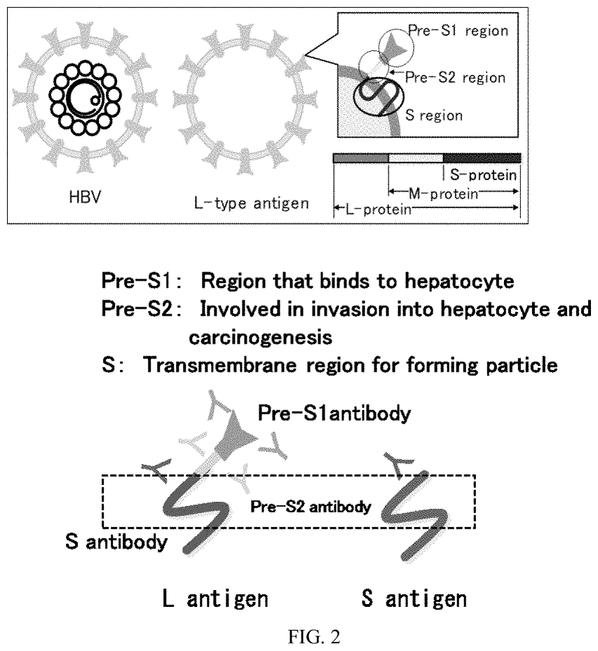

Three types of proteins, namely, HBs-L antigen, HBs-M antigen and HBs-S antigen, are present on the surface of a HBV virus particle (FIGS. 1 and 2). The HBs-L antigen is composed of three regions, i.e., Pre-S1 region, Pre-S2 region and S region, sequentially from the N-terminus of the protein displayed on the surface. The Pre-S1 region is a sensor region for HBV to recognize and bind to hepatocytes for infection, and plays an important role in the initial step of the HBV infecting mechanism. The Pre-S2 region is presumed to be involved in carcinogenesis, and is also considered to be responsible for invasion of HBV into the infected cell. Furthermore, the S region has a transmembrane domain that is crucial for HBV to maintain its structure as a virus particle. While the HBs-L antigen consists of three regions, the HBs-M antigen lacks the Pre-S1 region, and the HBs-S antigen consists only of the S region without the Pre-S1 and Pre-S2 regions. The L protein forming the HBs-L antigen is usually composed of 400 amino acids, but those with considerable number of deletions may be composed of, for example, 382 amino acids. If it consists of 400 amino acids, the Pre-S1 region consists of the 1st-119th amino acids from the N-terminus, the Pre-S2 region consists of the 120th-174th amino acids, and the S region consists of the 175th-400th amino acids. The crucial amino acid sequence in each region was conserved well across various variants, and the three regions can be distinguished easily even for those with considerable number of deletions. Currently available vaccines use the HBs-S antigen for the sake of manufacturing convenience. However, since the Pre-S1 region of the L protein is important for HBV to attach to the hepatocytes, it is desirable that an antibody or cell-mediated immunity against this region is induced.

Therefore, the present invention tried to develop a vaccine that shows a stronger effect of preventing onset of infection as compared to the currently available vaccines by immunizing with the HBs-L antigen (containing the amino acid sequence represented by SEQ ID NO:1) developed and manufactured by Beacle Inc. Beacle Inc. succeeded in stable mass production of a HBs-L antigen that consists only of the L protein, by replacing the 11 amino acids at the N-terminus of the Pre-S1 region with five signal peptides, and deleting the 163rd-168th amino acids (44th-49th amino acids of the Pre-S2 region).

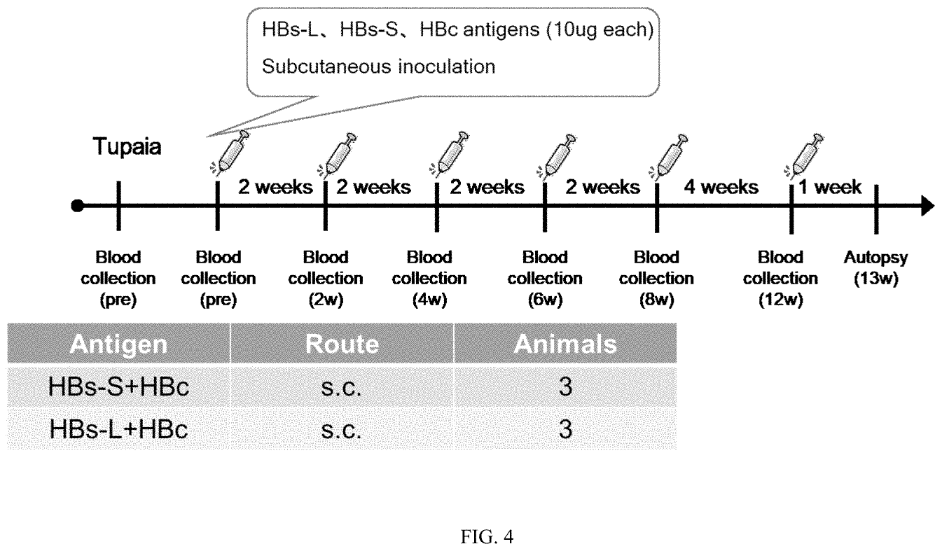

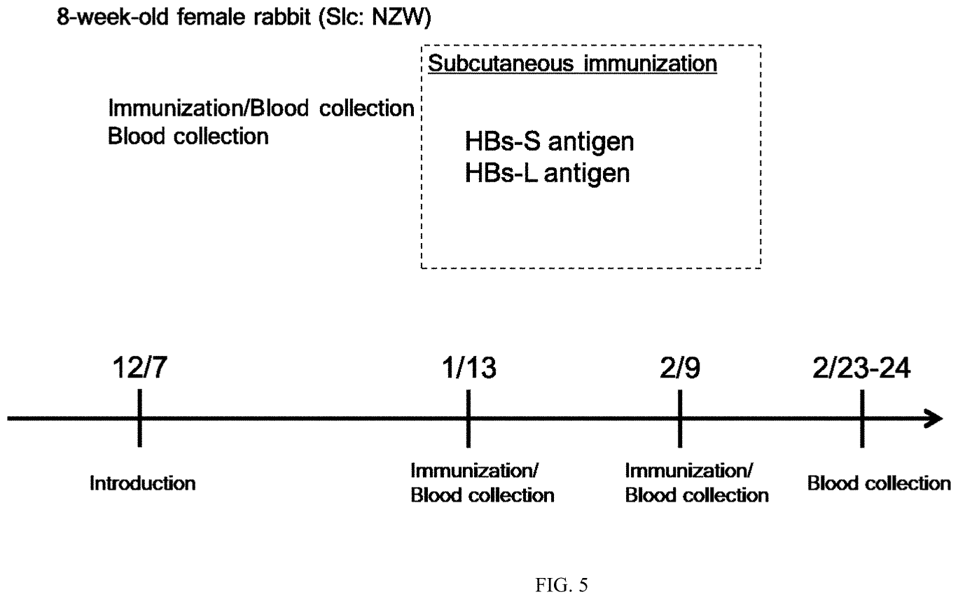

Small animal models susceptible to HBV, i.e., tupaia (FIG. 4) and rabbits (FIG. 5), were immunized with the HBs-L or HBs-S antigen to quantify and compare the antibody titers against the HBs-L or HBs-S antigen in their sera by ELISA.

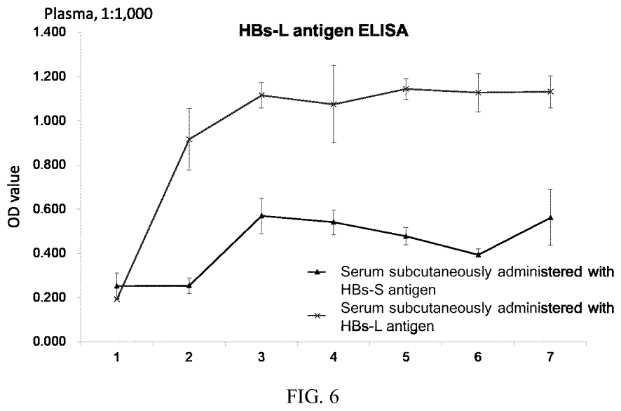

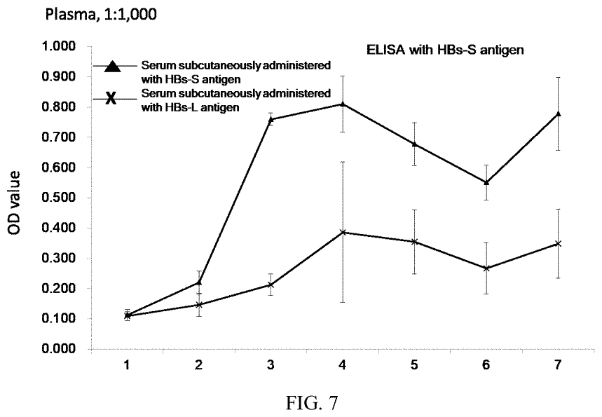

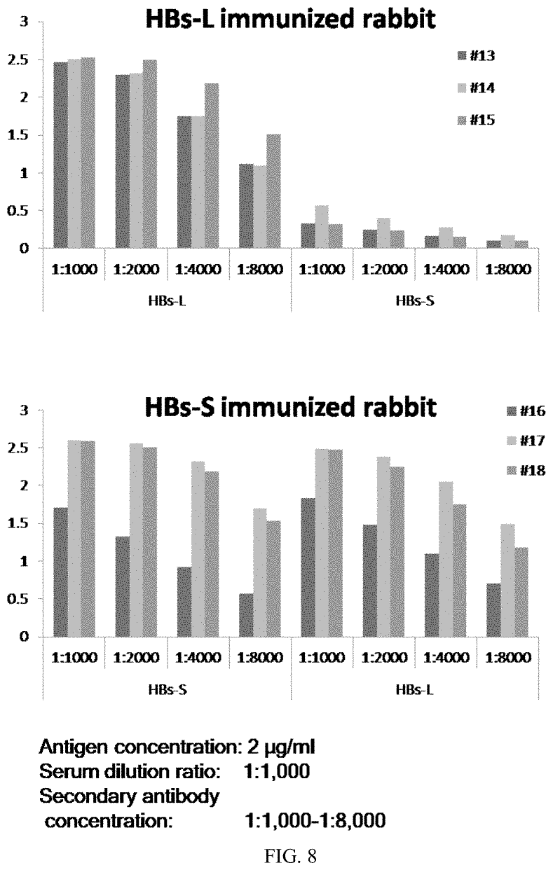

While an antibody that specifically bound to the HBs-L antigen was significantly produced in the sera of the animals immunized with the HBs-L antigen, an antibody that specifically bound to the HBs-S antigen was significantly produced in the sera of the animals immunized with the HBs-S antigen (FIGS. 6, 7 and 8).

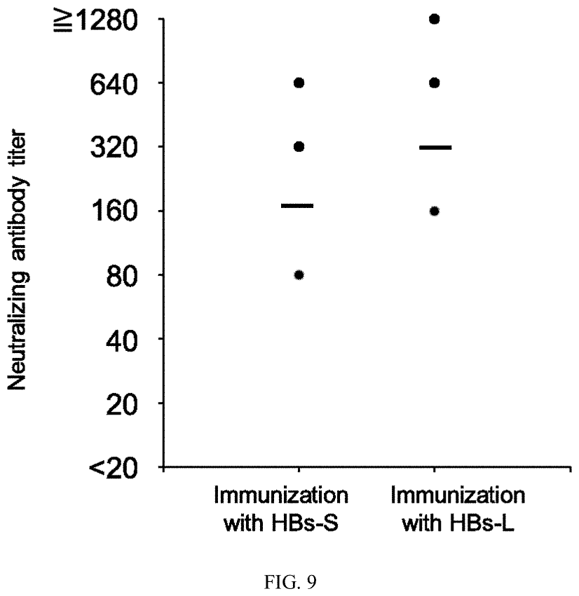

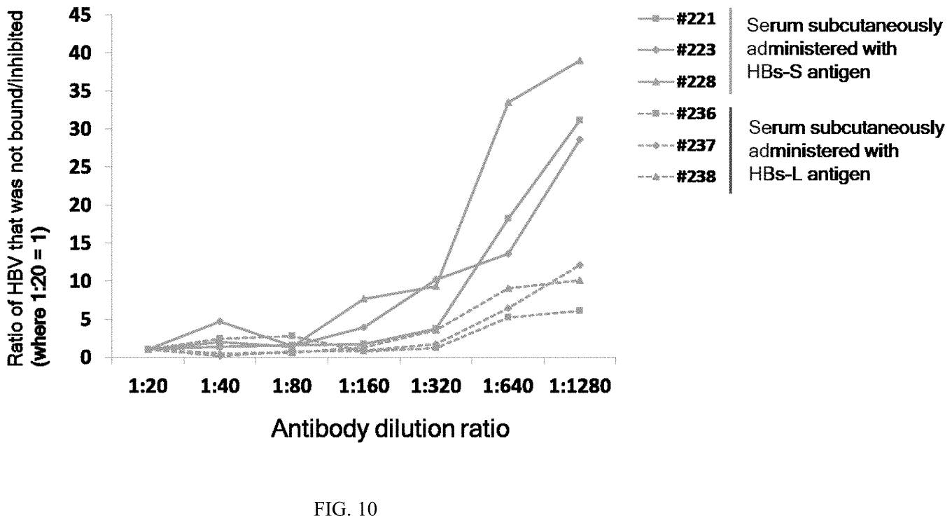

When the titers of the neutralizing antibodies in the sera immunized with the HBs-L or HBs-S antigen were compared and studied, the sera of the tupaia immunized with the HBs-L antigen indicated a higher neutralizing antibody titer (FIG. 9). Furthermore, in order to assess the binding strength of the antibody indicating this neutralizing activity, each of the sera was diluted and subjected to a neutralization test. As a result, the tupaia sera immunized with the HBs-L antigen showed stronger binding activity than those immunized with the HBs-S antigen (FIG. 10). Accordingly, a prophylactic vaccine using the HBs-L antigen was found to be superior than the currently available vaccines that use the HBs-S antigen.

Moreover, L antigen bound with an alum adjuvant was administered to a mouse to prepare a serum. Determination of the antibody titer in the serum showed that a Pre-S1 antibody was produced which had an antibody titer that was about 10 times higher than those of the Pre-S2 and S antibodies.

The present invention provides a hepatitis B vaccine comprising a surface antigen particle that has only the L protein among the L, M and S proteins of hepatitis B virus or a variant thereof assembling on a lipid membrane to form the particle.

Other than the L protein, the vaccine of the present invention may use a variant of the L protein. Examples of the L protein or a variant thereof of the present invention include:

(a) a protein comprising the amino acid sequence represented by SEQ ID NO:1; and

(b) a protein comprising an amino acid sequence obtained by deleting or substituting a total of 16 or less amino acids of the amino acid sequence represented by SEQ ID NO:1, which are: 6 or less amino acids in the Pre-S1 region represented by the 6th-113rd amino acids; 6 or less amino acids in the Pre-S2 region represented by the 114th-162nd amino acids; and 13 or less amino acids in the S region represented by the 163rd-385th amino acids.

The protein (b) above is a protein that functions as the L antigen. A “protein that functions as the L antigen” refers to a protein that functions as a vaccine so that an animal produces an antibody upon inoculation with the L antigen, and said antibody has a neutralizing activity against hepatitis B virus.

In addition, a protein that has an amino acid sequence obtained by deletion, substitution or addition of one or several amino acids in the amino acid sequence represented by SEQ ID NO:1, and that functions as the L antigen can also be used in the present invention. Examples of the amino acid sequence of such a protein include:

(i) an amino acid sequence obtained by substituting MGGWSSKPRKG (SEQ ID NO:6) for the 1st-5th amino acids KVRQG (SEQ ID NO:5) of the amino acid sequence represented by SEQ ID NO:1;

(ii) a sequence obtained by inserting 6 amino acids SIFSRT (SEQ ID NO:7) between the 156th and 157th amino acids of the amino acid sequence represented by SEQ ID NO:1;

(iii) a sequence having substitution of 13 or less amino acids in the S region represented by the 163rd to 385th amino acids of the amino acid sequence represented by SEQ ID NO:1; and

(iv) a sequence obtained by deleting or substituting a total of 16 or less amino acids of the amino acid sequence represented by SEQ ID NO:1, which are: 6 or less amino acids in the Pre-S1 region represented by the 6th-113rd amino acids; 6 or less amino acids in the Pre-S2 region represented by the 114th-162nd amino acids; and 13 or less amino acids in the S region represented by the 163rd-385th amino acids (excluding insertion of 6 amino acids between the 156th and 157th amino acids).

According to the present invention, a method for producing the L protein and a variant thereof is not particularly limited, and may be any method well known to those skilled in the art including synthesis by genetic engineering using a yeast or the like.

In order to synthesize the L protein by genetic engineering, first, DNA coding for this L protein is designed and synthesized. Such design and synthesis may be carried out, for example, by PCR method using a vector containing a gene coding for the L protein as a template, and primers designed to synthesize the DNA region of interest. Then, this DNA is linked to a suitable vector to obtain a recombinant vector for protein expression, and this recombinant vector is introduced into a host to express the gene of interest, thereby obtaining a transformant (Sambrook J. et al., Molecular Cloning, A Laboratory Manual (4th edition) (Cold Spring Harbor Laboratory Press (2012)).

In order to prepare the above-described variant protein, a mutation is introduced into the gene (DNA) coding for said protein. Mutation can be carried out by constructing an expression vector based on information of a gene having a mutation, or by using a mutation kit utilizing site-directed mutagenesis like Kunkel method or Gapped duplex method, for example, QuikChange™ Site-Directed Mutagenesis Kit (manufactured by Stratagene), GeneTailor™ Site-Directed Mutagenesis System (manufactured by Invitrogen) or TaKaRa Site-Directed Mutagenesis System (Mutan-K, Mutan-Super Express Km, etc.: manufactured by Takara Bio).

The host used for transformation is not particularly limited as long as it is capable of expressing the gene of interest. Examples of the host include yeasts, animal cells (COS cells, CHO cells, etc.), insect cells and insects. The method for introducing a recombinant vector into a host is known.

Then, this transformant is cultured to collect the L protein that is to be used as an antigen from the culture. A “culture” may refer to any of (a) culture supernatant, or (b) a cell culture, microbe cells, or a disrupted product thereof.

Subsequent to the cultivation, the host is disrupted to extract the L protein if the L protein of interest is produced inside the host. Alternatively, if the L protein is produced outside the host, the culture solution is used as it is or the host is removed by centrifugation or the like. Thereafter, a biochemical method generally employed for isolation/purification of a protein, for example, ammonium sulfate precipitation, gel filtration, ion-exchange chromatography, affinity chromatography or the like, can be employed alone or in an appropriate combination to isolate/purify L protein.

According to the present invention, the L protein can also be obtained by in vitro translation using a cell-free synthetic system. In this case, two types of methods, namely, a method using RNA as a template or a method using DNA as a template (transcription/translation), can be used. As the cell-free synthetic system, a commercially available system such as Expressway™ system (Invitrogen) can be used.

Moreover, the L protein used in the present invention has a self-assembling ability and thus is capable of displaying an antigen by assembling on the lipid membrane to form a particle. Specifically, all of the S, M and L proteins have a highly lipophilic S region, and all of the proteins are embedded in the lipid membrane when produced using a biological cell. Accordingly, the protein forms a stable antigen particle structure, and has high immunogenicity due to this particle structure. Examples of the method for displaying the antigen as such include methods described in Japanese patent Nos. 4085231 and 4936272.

Furthermore, according the present invention, other than mixing the core protein of hepatitis B virus with the aforesaid particles, it may be contained on the surface of or inside the L protein particles. The method for producing the core protein may utilize a conventionally reported method such as those described in Non-patent documents (for example, Rolland et al. J Chromatogr B Biomed Sci Appl. 2001 25; 753(1):51-65).

A vaccine obtained by the present invention produces an antibody against the Pre-S1 and/or PreS2 region of L protein upon administration to the subject. It also induces cell-mediated immunity against the Pre-S1 and/or PreS2 region of the L protein upon administration to the subject. Furthermore, a vaccine containing the core protein induces an antibody against the core protein or induces cell-mediated immunity against the core protein upon administration to the subject. Antibody induction can be confirmed by ELISA or the like. Herein, “cell-mediated immunity” refers to an immune system in which phagocytes, cytotoxic T cells, natural killer cells or the like are responsible for the elimination of foreign substances in the body.

At this point, the titer of the neutralizing antibody against hepatitis B virus is at least 2 to 1000, while the effect of inhibiting the binding of hepatitis B virus to human hepatocytes is at least 50-100%.

The vaccine of the present invention can be introduced into a living body by any known method, for example, intramuscular, intraperitoneal, intradermal or subcutaneous injection, nasal, oral or pulmonary inhalation, or oral administration. Furthermore, the HBs-L antigen contained in the vaccine of the present invention can be used in combination with an existing antiviral drug (for example, interferon). Since the way of the combinational use is not particularly limited, the vaccine of the present invention and an existing vaccine or antiviral drug may be introduced into a living body by administering them at the same time, or by administering either one of them after the other after a certain period of time.

Moreover, the vaccine of the present invention can be used as a vaccine composition by mixing it with a known pharmaceutically acceptable carrier such as an excipient, a filler, a binder or a lubricant, a buffer, a tonicity agent, a chelating agent, a colorant, a preservative, an aromatic agent, a flavoring agent, a sweetener or the like.

The vaccine composition of the present invention can be administered either orally or parenterally depending on whether it is an orally administered agent such as a tablet, a capsule agent, a powdery agent, a granular agent, pills, a liquid agent or a syrup agent, or a parenterally administered agent such as an injectable agent, a spray agent, an external agent or a suppository. Preferable examples include local injections such as intradermal, subcutaneous, intramuscular and intraperitoneal injections or nasal spray.

While the dose of the vaccine or the vaccine composition can appropriately be determined according to the type of the active component, the administration route, the administration target, age, weight, sex, symptoms or other conditions of the patient, the daily dose of HBs-L antigen is about 5-400 micrograms and preferably about 10-100 micrograms in the case of subcutaneous injection, and about 5-400 micrograms and preferably about 10-100 micrograms in the case of nasal spray. The vaccine or the vaccine composition of the present invention may be administered once or in several times a day.

### EXAMPLES

Hereinafter, the present invention will be described in more detail by way of examples. The scope of the present invention, however, should not be limited to these examples.

### Example 1

### Production of L Antigen

In this example, virus-like particles resulting from assembly of the L protein (having a self-assembling ability and consisting of the amino acid sequence represented by SEQ ID NO:1) on a lipid membrane were used as the L antigens, which was prepared according to the method described in the specification of Japanese Patent No. 4085231. Specifically, a yeast expressing the L antigen was prepared according to the method described in the specification of Japanese Patent No. 4085231. This yeast was cultured and then the cell culture was disrupted with glass beads according to the method described in the specification of Japanese Patent No. 4936272. The disrupted cell solution was subjected to a heat treatment at 70° C. for 20 minutes. Following the heat treatment, the resultant was subjected to a centrifugation process to collect the resulting supernatant. Subsequently, the collected supernatant was purified using a cellufine sulfate column and a gel filtration column, and concentrated to a protein concentration of 0.2 mg/mL or more to obtain L antigen.

### Example 2

### Biochemical/Physicochemical Properties of L Antigen

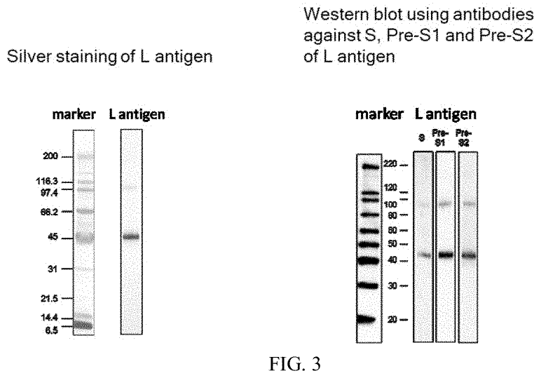

When the produced L antigen was subjected to electrophoresis and silver staining, a band indicating a monomer of the L antigen appeared near 45 kDa as shown in the left panel of FIG. 3, while a band indicating a dimer of the L antigen can be observed at a position of a molecular weight twice as much as said molecular weight. Meanwhile, when western blot was conducted to detect the L antigen, bands were observed at a position near 45 kDa as well as at a position of a molecular weight twice as much as said molecular weight with any of the S, Pre-S1 or Pre-S2 antibody, as shown in the right panel of FIG. 3.

The particle size of the L antigen was measured by dynamic light scattering method using Zetasizer (Malvern). As a result, the particle size was 59.7 nm, indicating that the antigen had a formed particle. Here, while the particle size is about 20 nm with a microscope that measures in a dry state, the particle size becomes greater with this system since the size is measured in an aqueous solution.

These results indicate that the L antigen did not contain a S protein or a M protein, that it was an antigen that consisted only of a L protein, and that it formed a particle.

### Example 3

### Preparation of thioredoxin-fused Pre-S1 and Pre-S2

A DNA fragment of Pre-S1 or Pre-S2 region was prepared from pGLD-LIIP39-RcT containing HBsAg L protein gene (Kuroda et al, J Biol Chem, 1992, 267: 1953-1961). The resulting DNA fragment was inserted into BamHI site of pET-32a (Novagen) to obtain expression vectors pET-32a-Pre-S1 and pET-32a-Pre-S2. These expression vectors were transformed into an E. coli expression strain BL21(DE3)pLysS to obtain an expressing strain. IPTG (isopropyl-β-thiogalactopyranoside) was added to the culture solution to induce expression of the cells.

The expressed cells were disrupted by ultrasonication to extract the protein, which was allowed to run through a Ni column (Chelating Sepharose Fast Flow, GE Healthcare) while increasing the imidazole concentration to elute Pre-S 1-TRX protein and Pre-S2-TRX protein.

After the purified product was dialyzed against PBS (phosphate buffered saline), the resultant was stored in a frozen state. Here, the protein concentration was measured using BCA Protein Assay Kit (Thermo).

### Example 4

### Antibody Production Upon Administration of L Antigen

L antigen was bound to an alum adjuvant. The resultant was administered to mice (ICR, Charles River Laboratories International, n=3) for three times every two weeks for 5 μg L antigen per mouse. Blood was collected four weeks after the final administration to prepare sera.

The Pre-S 1, Pre-S2 and S antibodies in the sera were determined as follows. The serum specimen was applied to an ELISA plate that had an S antigen (adr-type S antigen particles, Beacle Inc) immobilized thereon so as to use the S antibody bound to the antigen as the anti-S antibody, while using HRP-labeled anti-mouse IgG as the secondary antibody. In order to quantify the S antibody, commercially available mouse anti-S antigen monoclonal antibody (HBS, EXBIO) was used as the standard antibody for the calibration curve.

The Pre-S 1 antibody was determined as follows. Specifically, Pre-S 1-TRX protein prepared in Example 3 was immobilized on a ELISA plate and the rest was carried out in the same manner as the determination of the S antibody.

Here, Pre-S1 monoclonal antibody (Anti-HBs Pre-S1, mono 1, Beacle Inc.) was utilized as the standard antibody for the calibration curve. Similar to the case of Pre-S 1, the Pre-S2 antibody was measured by utilizing an ELISA plate that had Pre-S2-TRX protein immobilized thereon. Pre-S2 monoclonal antibody (2APS42, Institute of Immunology) was utilized as the standard antibody for the calibration curve. The obtained results are shown in Table 1.

TABLE 1
Antibody (ng/mL)

| anti-S | anti-Pre-S2 | anti-Pre-S1 |
|---|---|---|
| 599 | 447 | 5799 |

As can be appreciated from Table 1, production of the Pre-S1 antibody was found to be ten times as much as that of the Pre-S2 or S antibody, upon administration of L antigen. This indicated that the L antigen that consists only of the L protein was a suitable antigen for mass-producing the Pre-S 1 antibody.

### Example 5

Rabbits and small animal models susceptible to HBV, i.e., tupaia, were immunized with HBs-L or HBs-S antigen, and blood was collected after lapse of days (FIGS. 4 and 5). The antibody titers against the HBs-L or HBs-S antigen in their sera were quantified and compared by ELISA. Moreover, cell cultures susceptible to HBV infection were used to quantify the effect of inducing the neutralizing antibody. Furthermore, peripheral blood was collected from tupaia, a small animal model susceptible to HBV infection, after lapse of days and stored in a frozen state. These cells were used to quantify and compare the effect of inducing the cell-mediated immunity.

1) Virus

Hepatitis B virus (HBV) genotype C (C_JPNAT) was used. Primary human hepatocyte culture (PXB cells; PhoenixBio) was infected with this virus, and a culture supernatant of cells that resulted viral proliferation was used as a viral solution.

2) Virus and cells For experimental viral infections, HepG2-NTCP30 cells obtained by introducing and expressing human NTCP gene in HepG2 cells were used. In order to culture the HepG2-NTCP30 cells, 10 mM HEPES, 10% heat-inactivated fetal calf serum (Fetal Calf Serum: FCS), 5 μg/ml of insulin, 1 μg/ml of puromycin, 100 units/nil of penicillin and 100 μg/ml of streptomycin were added to Dulbecco's Modified Essential Medium/F12-Glutamax (Thermo Fisher) to use the resultant as a proliferation medium.

3) Animals

Tupaia (Tupaia belangeri) were purchased from Kunming Institute of Zoology, Chinese Academy of Sciences, and bred in-house to use the resulting individuals. Rabbits were 6-week-old Slc:NZW (Japan SLC).

4) Immunogens

For immunization of each animal, HBs-S, HBs-L and HBc antigens (Beacle Inc.) were used.

5) Immunization of Animals

For the tupaia, the HBs-S or HBs-L protein and the HBc protein were diluted to 100 μg/ml in phosphate buffered saline (PBS) to give an antigen solution. Three tupaia each were inoculated subcutaneously on the back with 100 μg/ml of the resulting antigen solutions, respectively. Immunization was conducted every two weeks for five times, and then immunization was once again conducted after four weeks. Blood was collected upon immunization as well as a week after the final immunization, for which EDTA blood collection tubes were used. Blood was centrifuged at 2,000 rpm for 10 minutes to separate the plasma. The plasma was stored at −80° C. until use.

Initial immunization for the rabbits was conducted by mixing the HBs-S protein (1 mg/ml) or the HBs-L protein (1 mg/ml) with an equivalent amount of Freund's complete adjuvant (Wako), and inoculating three rabbits each subcutaneously on the back with 100 μl of the resulting antigen solutions, respectively. Second immunization was conducted after a month, where incomplete adjuvant (Wako), instead of Freund's complete adjuvant, was mixed with the protein solution to prepare an antigen solution and used to subcutaneously inoculate on the back of the rabbits. Blood was collected a month after the immunization. The blood was centrifuged at 15,000 rpm for 10 minutes to separate the sera. The sera were stored at −80° C. until use.

6) Detection of anti-HBs antibody in tupaia specimens by ELISA antibody detection

The HBs-S or HBs-L protein was diluted in a 0.05M Na₂CO₃ carbonate buffer (pH 9.6) to 2 μg/ml to obtain an antigen solution as a capturing antigen, and 50 μl of which was dispensed into each well of a 96-well plate and incubated at 4° C. overnight. Subsequently, 100 μl of a blocking buffer (1% bovine serum albumin, 0.5% Tween, 2.5 mM EDTA in PBS) was added to each well and incubated at 37° C. for 2 hours for blocking. The resultant was washed with 200 μl of 0.5% Tween in PBS (PBST) for three times, and 50 μl of plasma 1,000-fold diluted in the blocking buffer was added to each well and incubated at 37° C. for 2 hours.

Then, following washing with 200 μl of PBST for three times again, 50 μl of anti-tupaia IgG rabbit antibody diluted in a blocking buffer to 1 μg/ml was added to each well as a secondary antibody, and incubated at 37° C. for 2 hours. Subsequently, following washing with 200 μl of PBST for three times, 50 μl of anti-rabbit IgG donkey antibody 10,000-fold diluted in a blocking buffer was added to each well as a tertiary antibody, and incubated at 37° C. for 2 hours. After washing with 200 μl of PBST for three times, 100 μl of a solution obtained by dissolving 40 mg of o-phenylenediamine dihydrochloride (OPD) in 10 ml of a 0.15M citrate buffer and adding 4 μl of hydrogen peroxide (H202) thereto was added to each well. After allowing the resultant to develop a color at room temperature for 10 minutes, 50 μl of 2M H₂SO₄ was added as a reaction terminator to each well to determine the absorbance at 492 nm.

7) Neutralization test

(1) Preparation of cells

HepG2-NTCP30 cells were used for the neutralization test. 250 μl of the cells at 2.0×10⁵ cells/ml were plated in each well of a collagen-coated 48-well plate. After culturing at 37° C. for 24 hours, the medium was replaced with a proliferation medium supplemented with 3% DMSO. Cells further cultured at 37° C. for 24 hours were used for the neutralization test.

(2) Procedure of Neutralization Test

The serum/plasma samples as the specimens were 10-fold diluted in a proliferation medium, and were further subjected to a two-fold serial dilution. These serum/plasma samples and a proliferation medium as a control were mixed with an equal amount of a viral solution that was prepared to have 6.0×10⁶ copies/ml, and the resultants were left to stand at 37° C. for an hour to allow reaction. At the end of the reaction, the HepG2-NTCP30 cells plated in the 48-well plate was inoculated with 125 μl of the mixture in each well, and the resultants were left to stand at 37° C. for 3 hours to allow reaction. At the end of the reaction, the mixture used for inoculation was removed, and 125 μl of a proliferation medium was poured into each well for washing for five times. After the washing, the cells were collected with large orifice tips and stored in a frozen state at −80° C. until use.

(3) Quantification of Virus Gene

Genes were extracted from the frozen cells using SMI TEST EX-R & D (Nippon Genetics). The virus gene was quantified by real-time PCR assay. 30 μl of the PCR reaction solution contained the gene for 250 ng, forward primer HB-166-S21 (nucleotides [nts] 166-186; 5′-CACATCAGGATTCCTAGGACC-3′ (SEQ ID NO:2)) for 6 pmol, reverse primer HB-344-R20 (nts 344-325; 5′-AGGTTGGTGAGTGATTGGAG-3′ (SEQ ID NO:3)) for 6 pmol, TaqMan probe HB-242-S26FT (nts 242-267; 5′-CAGAGTCTAGACTCGTGGTGGACTTC-3′ (SEQ ID NO:4)) for 9 pmol, and Thunderbird Probe qPCR Mix (Toyobo) for 15 μl. The PCR cycle included reactions at 50° C. for 2 minutes and 95° C. for 10 minutes, followed by 53 cycles of reactions at 95° C. for 20 seconds and 60° C. for a minute.

(4) Determination of Titer of Neutralizing Antibody

The quantity of the virus gene in each of the cell samples was determined to compare with the quantity of the virus gene in the control sample. Samples in which the quantity of the virus gene was 10% or less as compared to the gene in the control sample were assumed to have positive neutralization response against the antibody, and thus were found to be positive neutralizing antibodies. The titer of the neutralizing antibody was defined as the reciprocal of the highest dilution fold of the plasma/serum that showed neutralization response.

Results

Three types of proteins, namely, HBs-L antigen, HBs-M antigen and HBs-S antigen, exist on the surface of a HBV virus particle (FIGS. 1 and 2). Currently available vaccines use the HBs-S antigen for the sake of manufacturing convenience. However, since the N-terminus of the L antigen is responsible for HBV to attach to the hepatocytes, it is desirable that an antibody or cell-mediated immunity against this region is induced. Therefore, the present invention tried to develop a vaccine that shows a stronger effect of preventing onset of infection as compared to the currently available vaccines by immunizing with the HBs-L antigen (Ref. 2) developed and manufactured by Beacle Inc. Small animal models susceptible to HBV, i.e., tupaia (FIG. 4) and rabbits (FIG. 5), were immunized with the HBs-L or HBs-S antigen, and the antibody titers against the HBs-L or HBs-S antigen in their sera were quantified and compared by ELISA and based on the titer of the neutralizing antibody.

While an antibody specifically binding to the HBs-L antigen was significantly produced in the sera of the animals immunized with the HBs-L antigen, an antibody specifically binding to the HBs-S antigen was significantly produced in the sera of the animals immunized with the HBs-S antigen (FIGS. 6, 7 and 8). When the titers of the neutralizing antibodies in the sera immunized with the HBs-L or HBs-S antigen were compared and studied, the sera of the tupaia immunized with the HBs-L antigen indicated a higher neutralizing antibody titer (FIG. 9). Furthermore, in order to assess the binding strength of the antibody indicating this neutralizing activity, each of the sera was diluted and subjected to a neutralization test. As a result, the tupaia sera immunized with the HBs-L antigen showed stronger binding activity than those immunized with the HBs-S antigen (FIG. 10).

Accordingly, a prophylactic vaccine using the HBs-L antigen was shown to be superior than the currently available vaccines using the HBs-S antigen.

Discussion

When tupaia and rabbits were immunized with the HBs-L antigen, antibodies against the Pre-S1 or Pre-S2 region that specifically reacted with the HBs-L antigen and that had less cross-reactivity with the HBs-S antigen was mainly produced, in addition to the HBs-S antibody. Furthermore, the tupaia sera immunized with the HBs-L antigen showed higher neutralizing antibody titers and stronger binding activity than those immunized with the HBs-S antigen. Since the Pre-S1 or Pre-S2 region is used by hepatitis B virus to attach and invade into the hepatocytes, induction of an antibody or cell-mediated immunity against this region is expected to have a stronger prevention effect against the infection than the currently available vaccines.

Moreover, also as a therapeutic vaccine for hepatitis B (Refs.3 and 4), use of the HBs-L antigen is considered to inhibit attachment/invasion of the virus into the hepatocytes by the action of the antibody or the cell-mediated immunity against the Pre-S1 or Pre-S2 region, together with the antibody against the HBs-S antigen, and thus it can be used as an antiviral therapeutic that can induce hepatitis B virus antigen/antibody seroconversion.

Use of the HBs-L antigen as a universal vaccine can reduce the number of HB vaccine non-responders, which may possibly lead to stronger infection prevention of hepatitis B virus and even more to eradication of hepatitis B. In addition, use of the HBs-L antigen as a therapeutic vaccine can solve the problems associated with currently available therapies such as nucleotide analog formulation or interferon, and may offer good news for hepatitis B patients as a novel antiviral therapy that can induce hepatitis B virus antigen/antibody seroconversion.

### Example 6

Production of C-Antigen

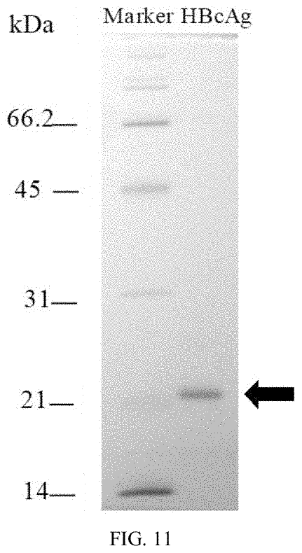

Full-length DNA of HBcAg (ACC#X01587) was inserted into pET-19b vector that had been removed of sequences such as His-tag to prepare an expression vector for HBcAg. The resulting expression vector was introduced into E. coli to obtain an expressing strain. The E. coli strain was cultured to obtain bacterial cells. The resulting bacterial cells were disrupted, and the supernatant thereof was subjected to ammonium sulfate precipitation. The precipitate was dissolved and the resultant was subjected to sucrose density gradient centrifugation to obtain a HBcAg fraction. This fraction was passed through gel filtration column to purify HBcAg. The purified HBcAg presented a 21 kDa single band upon silver staining following electrophoresis (FIG. 11). Where HBcAg core proteins are known to bind to each other to form a particle, the particle size was 45 nm as measured by dynamic light scattering method using Zetasizer (Malvern), showing that a particle was formed.

### Example 7

Mouse Antibody Detection by ELISA (HBs-S, HBs-M, HBs-L Antigen Administration)

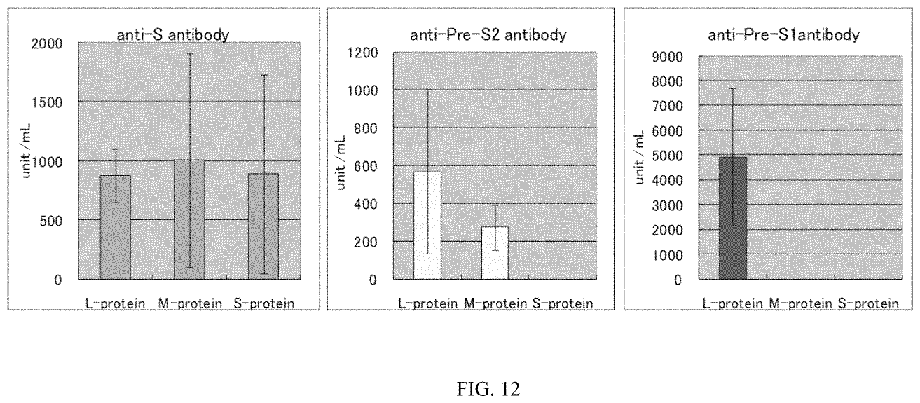

The HBs-S, HBs-M or HBs-L antigen was administered to mice to see the binding of them to Pre-S1 peptide, Pre-S2 peptide and HBs-S antigen to determine the amount of the antibodies against the Pre-S1, Pre-S2 and S antigen. As a result, the Pre-S1 antibody was produced only when the L antigen was administered (FIG. 12). In addition, about 80% of the antibodies were the antibody against Pre-S1.

Since Pre-S1 is a region that recognizes the hepatocytes when HBV infects human hepatocytes, if the antibody against this region really has an effect of preventing HBV infection, the L antigen would have stronger prevention action against HBV infection.

### Example 8

Test for Activation of Cell-Mediated Immunity Upon Administration of HBs-L and HBc Antigens

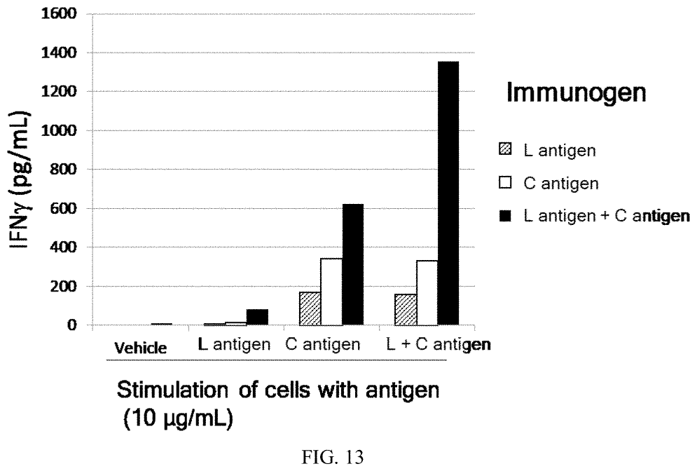

Spleen cells from mice immunized with HBs-L antigen, HBc antigen, and HBs-L+HBc antigen were stimulated with the antigens to observe activation of cell-mediated immunity (INF-'γ increase) (Table 2, FIG. 13).

TABLE 2

| | Stimulation of cells with antigen (10 μg/mL) | | | |
|---|---|---|---|---|
| Immunogen | Vehicle | L antigen | C-antigen | L + C-antigen |
| L antigen | 0.00 | 8.32 | 169.71 | 157.00 |
| C-antigen | 0.00 | 13.02 | 342.76 | 332.00 |
| L antigen + | 9.50 | 83.33 | 623.60 | 1353.33 |
| C-antigen | | | | |

The cell-mediated immunity of the mice immunized with the HBs-L antigen was hardly activated upon stimulation with the L antigen. The cell-mediated immunity of the mice immunized with the HBc antigen was activated upon immunization with the HBc antigen and the HBs-L+HBc antigens. The cell-mediated immunity of the mice immunized with the HBs-L+HBc antigens was activated in every cases, and strongly activated upon stimulation with the HBs-L+HBc antigens. These results showed that the cell-mediated immunity was strongly activated by immunization with a mixture of the HBs-L antigen and the HBc antigen.

### Example 9

L Antigen Safety Study

A non-GLP single-dose intravenous administration toxicity study was conducted for the L antigen using rats (5 cases per group). A solvent, i.e., phosphate buffered saline, as the control group, and the L antigen at doses of 0.2, 1 and 5 mg/kg were administered, as a result of which none of the groups exhibited abnormal general state and had no fatalities. In addition, no abnormality was observed in the weight change or autopsies. Therefore, the maximum tolerated dose was suggested to be more than 5 mg/kg. A non-GLP repeated intravenous administration toxicity study was conducted for the L antigen for 28 days using rats (6 cases per group). A solvent, i.e., phosphate buffered saline, as the control group, and the L antigen at doses of 0.05 and 0.25 mg/kg were administered once a day for 28 days, as a result of which none of the groups exhibited abnormal general state and had no fatalities. In addition, no abnormality was observed in the weight change. However, increases in the spleen weight and white blood cell count were observed. These abnormality presumably resulted from the immune response caused by the repeated L antigen administrations. Therefore, the maximum dose of non-observed effect level in terms of toxicology was suggested to be more than 0.25 mg/kg.

### RELATED INFORMATION AND PAPERS

1) Japanese Patent No. 4085231

2) Sanada T, Tsukiyama-Kohara K, Yamamoto N, Ezzikouri S, Benjelloun S, Murakami S, Tanaka Y, Tateno C, Kohara M., Property of hepatitis B virus replication in Tupaia belangeri hepatocytes., Biochem Biophys Res Commun., 2016 Jan 8; 469(2):229-35. doi: 10.1016/j.bbrc.2015.11.121.

3) Akbar S M, Al-Mahtab M, Jahan M, Yoshida O, Hiasa Y., Novel insights into immunotherapy for hepatitis B patients., Expert Rev Gastroenterol Hepatol. 10(2):267-76, 2016.

4) Fazle Akbar SM, Al-Mahtab M, Hiasa Y., Designing immune therapy for chronic hepatitis B., J Clin Exp Hepatol. 4(3):241-6, 2014.

### SEQUENCE LISTING FREE TEXT

SEQ ID NO:2: Synthetic DNA

SEQ ID NO:3: Synthetic DNA

SEQ ID NO:4: Synthetic DNA

1. A hepatitis B vaccine comprising a surface antigen particle that has only hepatitis B virus L protein or a variant thereof assembling on a lipid membrane to form the particle.

2. The vaccine according to claim 1, wherein the L protein or the variant thereof is protein (a) or (b) below:
(a) a protein comprising the amino acid sequence represented by SEQ ID NO:1; and
(b) a protein comprising an amino acid sequence obtained by deleting or substituting a total of 16 or less amino acids of the amino acid sequence represented by SEQ ID NO:1, which are: 6 or less amino acids in the Pre-S1 region represented by the 6th-113rd amino acids; 6 or less amino acids in the Pre-S2 region represented by the 114th-162nd amino acids; and 13 or less amino acids in the S region represented by the 163rd-385th amino acids.

3. The vaccine according to claim 1, wherein the L protein or the variant thereof is expressed in yeast.

4. The vaccine according to claim 1, which produces an antibody against the Pre-S1 and/or Pre-S2 region of the L protein upon administration to the subject.

5. The vaccine according to claim 1, which induces cell-mediated immunity against the Pre-S1 and/or Pre-S2 region of the L protein upon administration to the subject.

6. The vaccine according to claim 1, further comprising a core protein of hepatitis B virus.

7. The vaccine according to claim 6, which further induces an antibody against the core protein upon administration to the subject.

8. The vaccine according to claim 6, which further induces cell-mediated immunity against the core protein upon administration to the subject.

9. The vaccine according to claim 1, wherein the titer of the neutralizing antibody against hepatitis B virus is at least 2 to 1000.

10. The vaccine according to claim 1, whose effect of inhibiting the binding of hepatitis B virus to human hepatocytes is at least 50-100%.

11. A hepatitis B vaccine comprising an hepatitis B virus L protein or a variant thereof, which is obtained by a method of synthesizing and isolating and purifying by a genetic engineering method or a method of synthesizing by a cell-free synthetic system, wherein the L protein or a variant thereof has an antigenic particle structure.

12. The hepatitis B vaccine according to claim 11, wherein the method of synthesizing and isolating and purifying by the genetic engineering method is a method of producing a recombinant vector for expressing a protein having a DNA encoding the hepatitis B virus L protein or a variant thereof, introducing the vector into a host, culturing the obtained transformant, and isolating and purifying the protein.


SEQUENCE LISTING (7 sequences)

SEQ ID NO: 1 - protein, 388 aa, Hepatitis B virus
KVRQGMGTNLSVPNPLGFFPDHQLDPAFGANSNNPDWDFNPNKDQWPEANQVGAGAFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISGDPAPNMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 2 - DNA, 21 bp, Artificial
  Synthetic DNA
cacatcaggattcctaggacc

SEQ ID NO: 3 - DNA, 20 bp, Artificial
  Synthetic DNA
aggttggtgagtgattggag

SEQ ID NO: 4 - DNA, 26 bp, Artificial
  Synthetic DNA
cagagtctagactcgtggtggacttc

SEQ ID NO: 5 - protein, 5 aa, Hepatitis B virus
KVRQG

SEQ ID NO: 6 - protein, 11 aa, Hepatitis B virus
MGGWSSKPRKG

SEQ ID NO: 7 - protein, 6 aa, Hepatitis B virus
SIFSRT